(19) United States
(12) Patent Application Publication
(10) Pub. No.: US 2008/0243023 A1
(43) Pub. Date: Oct. 2, 2008

(54) Device For Preventing Bruxism

(76) Inventors: Cornelis Albert Valkhof, Eindhoven (NL); Joost Helena Petrus Maria Verdonk, s'-Hertogenbosch (NL)

Correspondence Address: YOUNG & THOMPSON, 209 Madison Street, Suite 500, ALEXANDRIA, VA, 22314, US

(21) Appl. No.: 11/630,515

(22) PCT Filed: Jun. 22, 2005
(86) PCT No.: PCT/NL2005/000454

§ 371 Date: Feb. 1, 2008

(30) Foreign Application Priority Data
Jun. 22, 2004 (NL) 1026474

(51) Int. Cl. A61B 5/04

(52) U.S. Cl. 600/546

(57) ABSTRACT

A device for preventing bruxism includes a carrier (1) which is intended for receiving in a mouth of a user and which includes at least a part of an electronic bio-feedback system. The bio-feedback system includes at least one signal sensor (2) which is sensitive to electrical muscle signals and which is coupled to a control unit. The control unit is designed and adapted to generate a correcting stimulus via an electrode (2) when a muscle signal is detected above a predetermined threshold level. The bio-feedback system is herein provided with elements for individually adjusting and storing the threshold level.

The present invention relates to a device for preventing bruxism, comprising a carrier intended for receiving in a mouth of a user, which carrier comprises at least a part of an electronic bio-feedback system, which bio-feedback system comprises at least one signal sensor which is sensitive to electrical muscle signals and which is coupled to a control unit designed and adapted to generate a correcting stimulus via an electrode when a muscle signal above a predetermined threshold level is detected.

Bruxism is an involuntary or subconscious para-function which takes place during the day as well as at night, and is manifested in a static or dynamic contact between the chewing surface complexes of the lower and upper jaw, wherein a pronounced neuromuscular activity occurs. These contacts do not form part of the normal functional physiology. Bruxism is also known as clenching and/or grinding of the teeth.

It is suspected that in the vast majority of cases bruxism is caused by stress resulting from suppressed flight response. Flight response is a normal instinctive phenomenon in nature, not only in animals but also in humans. During rest the parasympathetic part of the nervous system is active. A shock reaction disturbs this rest and will result in a flight response. There occurs here an acute shift from the parasympathetic part of the nervous system to the sympathetic part. Under the influence of hormones, such as (nor)adrenaline, cortisol, serotin and pheromones, blood is sent to the extremities to reinforce a flight response. When the flight attempt is successful or the danger has passed, the balance shifts back to the parasympathetic part of the nervous system and the rest state is eventually restored.

This instinctive behaviour still exists in humans, albeit that highly civilized man is able to suppress this flight response. This latter occurs for instance when someone finds himself in a traffic-jam on the way to an appointment. He/she wants to get away, but cannot do so. All physical phenomena linked to flight response now occur, but the eventual flight response is not effected. The sympathetic part of the nervous system is nevertheless in a full state of preparedness. At that moment the muscles of the head-neck area, i.e. the chewing muscles in particular, begin to play a crucial role.

Though tensioning of the chewing muscles a powerful biting action takes place, whereby small pain stimuli occur locally which can become manifest in, among other ways, pain in the teeth and molars, pain in the periodontium, pain in the jawbone around the roots of the teeth and molars, pain in the chewing muscles and pain in the jaw joint. These pain stimuli are per se hardly perceptible, but at a sufficient level will nevertheless result in suppressed flight response and the associated shift within the nervous system. The release of hormones induced by this flight response in turn causes tensioning of the chewing muscles, thereby resulting in a vicious circle which expresses itself in bruxism.

Bruxism is essentially a mechanical way of coping with stress which is experienced daily. In this respect bruxism is harmless and also functional. If however bruxism occurs too often this results in an increase of the chewing forces, whereby normal, intended chewing movements during eating are also carried out with so much force that pain stimuli occur which in turn induce the above stated vicious circle. In addition to stress, normal daily occurrences will thus also provoke suppressed flight response or bruxism. The chewing force can herein increase significantly so that as a result of bruxism teeth and molars can be damaged and even muscular complaints, headaches and shoulder/neck disorders can result. Jaw joint disorders can eventually also occur, so-called arthrogenic disorders such as cracking jaw joints, in addition to earache and balance disorders. When a patient experiences such a wide variety of complaints to a lesser or greater degree, bruxism no longer has a functional and harmless nature but by now a more destructive one.

Bruxism therefore has both direct and indirect adverse effects on the state of health of a person. Firstly, bruxism causes damage to the teeth which is directly discernible as such. Indirectly, bruxism however also has an adverse effect on the entire locomotor apparatus, i.e. muscles can tense up and joints can become overloaded or damaged. In addition, bruxism can cause disrupted sleep, with all the possible neurotic and psychological consequences this entails. The device according to the invention has for its object to avoid or at least significantly alleviate bruxism, and thereby the above described adverse consequences.

A device of the type stated in the preamble is known from European patent EP 1.110.518. The device described herein comprises a carrier in the form of a splint which is received in the whole mouth. Situated on the splint are two metal anchoring elements. These anchoring elements serve on the one hand as signal sensor or antenna with which electrical muscle signals can be received and recorded. The anchoring elements also serve on the other hand as electrode on which a correcting stimulus can be generated as soon as incipient bruxism is detected. With this in mind, the signal sensors and electrodes are incorporated in an appropriate bio-feedback system which is partially integrated in the splint. As soon as the antenna detects an unintended, non-physiological activity of the teeth, for instance when the user is sleeping, this signal is generated to a control unit of the bio-feedback system. When a certain threshold level is exceeded, the control unit actuates a pulse generator which in turn generates an electrical pulse and delivers this via the electrode as electrical stimulus to the jaw of the user in order to restore a state of rest therein. Bruxism can thus be nipped in the bud and the above stated adverse effects avoided. Bruxism will hereby decrease in the user and finally disappear completely as a result of an operant conditioning. The device can otherwise also be applied as a preventive measure, particularly in patients who already suffer from bruxism but who have not yet developed any harmful effects.

Although the known device is very effective per se in suppressing bruxism, the device is not always found to be suitable for all users. Particularly in the case of chronic bruxism patients the device thus occasionally generates no correcting stimulus at all, or does so only relatively late. The invention has for its object to provide an improvement in the device of the type stated in the preamble which thus becomes more widely usable and effective.

In order to achieve the stated object, a device of the type stated in the preamble is characterized according to the invention in that the bio-feedback system is provided with means for adjusting and storing the threshold level. The muscle activity detected by means of the signal sensor reflects the muscle activity of the user. The present invention is based on the insight that strongly developed muscles can suffice with a lower muscle activity than less developed muscles in order to produce a determined clenching or jaw pressure. The signal detected by means of the signal sensor will therefore be lower in the case of strong muscle groups than in the case of weaker muscles. Particularly users suffering from bruxism generally have strongly developed muscles, whereby the threshold value is occasionally not reached with the known device while an unacceptable jaw pressure is nevertheless being exerted. Owing to the present invention the threshold value is adjustable per device, whereby the threshold value in such a case can be adjusted downward so as to still enable delivery of the desired correction in good time. If on the other hand the device is employed for preventive use or in other manner in a user with relatively weakly developed jaw muscles, the threshold value can conversely be adjusted upward so that the device does not deliver a correcting stimulus prematurely. The device according to the invention can thus be individually modified in relatively simple manner to the specific situation of the user.

In a particular embodiment, the device according to the invention has the feature that the threshold value is adjustable to a level of between about 5 millivolts and 50 millivolts. It has been found that such an adjustment range is sufficient to enable effective adjustment of the device per user so as to thus reduce and eventually suppress in individual cases incipient and even advanced bruxism symptoms in the user.

Although the adjustment of the threshold level can be realized experimentally, for instance using a potentiometer, a variable capacitor or other electronic adjusting element, a preferred embodiment of the device according to the invention has the feature that the bio-feedback system is provided with means for placing the device in a learning mode in which an output signal from the signal sensor is stored as threshold signal. In this case the setting of the device takes place at least substantially by way of software. The device is placed for this purpose in a learning mode and subsequently measures a modal and maximum signal level in the jaw muscles of the user and sets the threshold value on the basis thereof. The device is then ready for use and, if desired, is at all times relatively simple to reset.

A further preferred embodiment of the device according to the invention is characterized in that the at least one signal sensor is able and adapted to detect a first muscle signal in a first frequency domain and a second muscle signal in a second frequency domain, and that the bio-feedback system comprises a threshold value per frequency domain. This embodiment is based on the insight that bruxism can manifest itself in two variants. In the first place there is so-called grinding of the teeth, which occurs mainly at night during sleep. In addition, bruxism is also manifested in clenching together of the jaws. It has been found that in such cases the muscle activity for these two mechanisms is measured for at least the most part at different frequencies. By setting a threshold value per frequency range and checking the activity against this value, the present preferred embodiment provides a remedy against both forms of bruxism. A further preferred embodiment is herein characterized more particularly according to the invention in that the first and second frequency domain lie respectively in a frequency range of about 1 to 20 Hz and about 90 to 600 Hz. It has been found that grinding of the teeth is measured particularly in the first frequency range, while clenching is detected particularly in the second frequency range.

It is found in practice that the jaw muscles practically always display a certain activity, which manifests itself in an output signal of the signal sensor. With more intense activity, particularly during bruxism, the output signal increases, which can be an indication that it is necessary to intervene. It is therefore not so much the absolute value of the signal, but much more the variation relative to the rest state which indicates whether bruxism is or is not (yet) occurring. In order to be able to identify this effectively, a further particular embodiment of the device according to the invention has the feature that the control unit comprises a processing unit which is able and adapted to determine a running average of a muscle signal measured during a rest state and to store this average as offset value. In this case the average rest activity can be measured and stored as offset value per individual. A measured muscle signal can then be compared thereto in order to establish whether there is an increased muscle activity. In a further particular embodiment, the device has the feature herein that the processing unit is able and adapted to decrease a measured muscle signal by the offset value in operating state and present this as usable signal to the control unit. The usable signal is thus compensated per individual for the individual rest activity, whereby the further processing by the bio-feedback system can take place with at least a high degree of uniformity.

It has been found that the device sometimes delivers a correcting stimulus too quickly; i.e. due to circumstances, for instance during a dream, a high jaw pressure is detected briefly without bruxism necessarily occurring. Bruxism is distinguished from such symptoms by a certain persistent jaw pressure. With this in mind, a further preferred embodiment of the device according to the invention has the feature that the control unit comprises clock means for generating a control signal only after a determined time period has elapsed during which the input signal lies above said threshold value. In this case the device does not respond immediately the threshold value is exceeded, but only after the optionally adjustable time period has elapsed. This avoids such premature correction signals, or at least reduces them considerably. In order to also allow individual variation of this parameter, a further particular embodiment herein has the feature that said time period is adjustable between 1 and 5 seconds.

Exceptionally good results have been achieved in practice with a further preferred embodiment of the device according to the invention which is characterized in that the processing unit comprises adding means which are able and adapted to find the sum of time periods during which said threshold value has been exceeded while subtracting intervening time periods during which said threshold value has not been reached. In this case the output signal is as it were balanced from the point where threshold value is exceeded, whereby brief peaks and valleys therein do not result in undesirable bio-feedback behaviour, or only do so in reduced measure.

The control unit is coupled to the carrier having at least a part of the bio-feedback system therein. If the threshold value is stored in or at the control unit, it is necessary to make sure that the control device in question is not connected to another carrier, since possibly incorrect or even harmful control signals can then be transmitted. With this in mind, a particular embodiment of the device according to the invention has the feature that the carrier is provided with an electronic element which is able to comprise an electronically detectable identification value associated with the carrier, and that the control device comprises read means which are able to record the identification value of a carrier coupled thereto and compare it to a value stored in the control device. The identification value thus provides a verification key, on the basis of which the control unit is able to determine with a sufficient degree of certainty that the correct carrier is connected.

A further embodiment of the device according to the invention more particularly has the feature herein that the electronic element is from a group comprising electrical resistors, capacitors and memory circuits which can at least be read out electronically. In the first two cases an almost unambiguous verification takes place by means of a comparison of resistance values or capacitance values from a countable series of applied values, while an electronically readable memory provides the option of storing a unique (digital) identification code therein for the purpose of an absolutely unambiguous correspondence.

It is important that the identification value is connected non-releasably to the carrier to enable at least sufficiently unique identification of the carrier at all times. A particular embodiment of the device according to the invention has for this purpose the feature herein that the carrier is connected by means of a cable to the control unit and that the electronic element is integrated in the cable, in particular in a connector thereof. Because the electronic element, which comprises the identification value, is thus incorporated in the cable connection to the carrier, the carrier can be uniformly produced and no electronic terminals and additional components need be arranged therein which would then be accommodated in a moist environment during use.

The invention will now be further elucidated on the basis of an exemplary embodiment and an associated drawing. In the drawing:

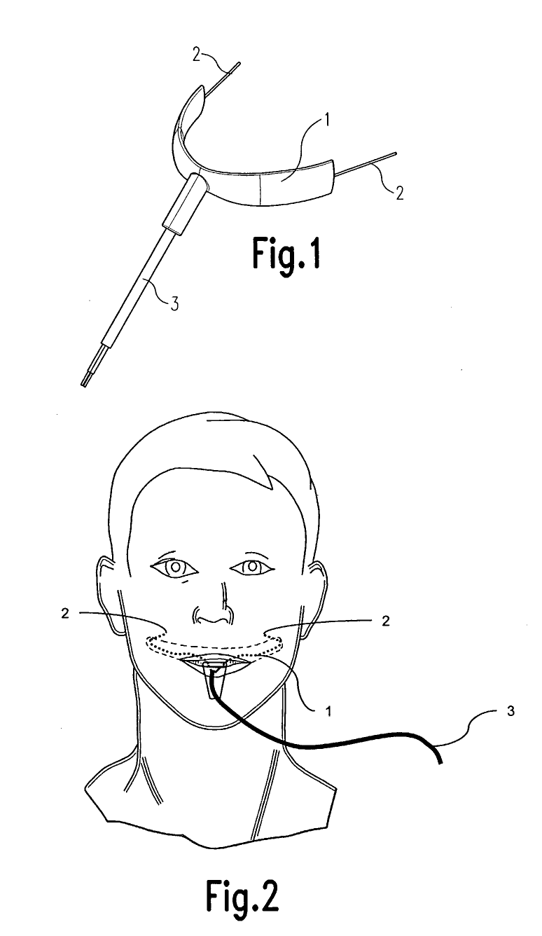

FIG. 1 shows a perspective view of an embodiment of a device according to the invention;

FIG. 2 shows the device of FIG. 1 placed in a user; and

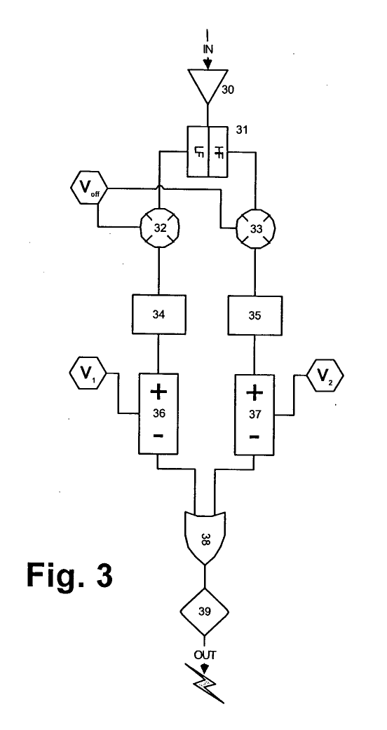

FIG. 3 shows a schematic operating diagram of the device of FIG. 1.

The figures are drawn purely schematically and not to scale. Some dimensions in particular may be exaggerated to greater or lesser extent for the sake of clarity. Corresponding parts are designated as far as possible in the figures with the same reference numeral.

FIG. 1 shows an exemplary embodiment of a device for preventing bruxism according to the invention. The device comprises a jaw-shaped carrier body 1 of a thermoplastic plastic, for instance a bio-compatible synthetic resin, from which protrude two metal antennas 2. The antennas herein extend on either side from the outer ends of the carrier body 1, but are otherwise embedded liquid-tightly in the material thereof so as to prevent corrosion or other destructive effect thereon.

Antennas 2 form anchoring members for fixing the carrier body against the teeth of the user on a buccal side, as shown schematically in FIG. 2. Carrier 1 thus rests at a fixed position against the teeth of the user. Antennas 2 are formed in this case from a wire with a solid core of bio-compatible material, in this example orthodontic steel, and can be cut to size relatively easily using a pair of pliers or other tool and bent into the desired shape round a jaw element using a similar tool. As shown in FIG. 2, the anchoring members are eventually situated at the position of jaw elements P2SD, P2SS, M1SD and M1SS, which guarantees good operation in practice, although fixing at other positions is also possible.

In addition to anchoring members for optimal fixation of carrier body 1 in the mouth of the user, the two antennas also form a signal sensor sensitive to electrical muscle signals. This signal sensor 2 forms part of a bio-feedback system of the device and is able to detect and generate as electronic signal (chewing) muscle activity in the jaw of the user. In order to measure the muscle activity, members 2 operate as a receiving antenna for wirelessly receiving an electro-muscular signal from the chewing muscles. The exact position of members 2 herefor is found in practice not to be very critical. Signal sensor 2 generates the recorded signal to a central control unit of the bio-feedback system which is accommodated in an external housing together with an electric power supply and a pulse generator and which is connected by means of an electric cable 3 to the signal sensor. The control unit comprises an integrated microprocessor which includes a processing unit and a non-volatile electronically rewritable and readable memory. The control unit receives signals from signal sensor 2 and, on the basis of a program code loaded therein, actuates the pulse generator for generating and delivering on command an electrical stimulus to the electrodes.

Carrier body 1 is preferably worn on the upper teeth, as shown in FIG. 2, wherein the connecting cable 3 protrudes out of the mouth. Because connecting cable 3 is connected to the buccal side of carrier 1, cable 3 does not impede full closing of the jaws. If desired, use can also be made of an alternative embodiment wherein a wireless connection is applied for the signal transfer, for instance based on so-called bluetooth or other standard protocol for wireless data transfer, and an electric power source is optionally integrated into the carrier. The embodiment described here is however found in practice to be already sufficiently ergonomic not to limit the user in his/her freedom of movement and not disturb his/her sleep. Cable 3 is connected by means of a connector (not shown) to a base station comprising the control unit. An electrical resistor is herein integrated into the connector which has a value chosen relatively at random from a countable series of for instance 50 different values. During synchronizing of the device this value is taken over in the control device and stored therein, so that by comparing the stored value to the connected value the control unit can then carry out a sufficiently reliable verification that the correct carrier is coupled thereto.

The control unit of the system is designed and adapted to transmit one or more electrical stimuli to the jaw of the user when a muscle activity above a predetermined threshold value is detected in order to induce an existing relax reflex therein. The chewing muscles then relax and possible bruxism is thus nipped in the bud. Such correcting stimuli are generated via the same antennas 2 as those with which the muscle signal has already been detected. Antennas 2 thus fulfil a multiple function within the bio-feedback system, i.e. in addition to being a signal sensor for recording a muscle activity, it is also an electrode for generating the correcting electrical stimulus. By administering an electrical pulse as stimulus at the correct position via electrodes 2, the detected para-functional muscle activity is interrupted and a relax reflex is induced, whereby the chewing muscles return to a state of rest. This is the same reflex which occurs when one bites unintentionally on something hard, for instance a hard piece in a currant bun.

Through wearing the device and receiving a correcting stimulus each time there are signs of bruxism, the user becomes conditioned, whereby the bruxism behaviour will eventually stop completely. By using the device to a sufficient extent and for a long period, bruxism can thus be overcome and the neuromuscular equilibrium of the head/neck area restored. The chewing force then has its normal physiological value again and the patient is rid of the above described adverse effects of bruxism. Because the vicious circle of bruxism also described above is broken and physiologically the body is functioning normally again, existing, already contracted disorders also have the opportunity to disappear.

For optimal operation it is important that the device does not deliver a correcting stimulus too early or too late. It has been found that the normal rest activity in the jaw muscles can vary greatly from user to user. Strongly developed jaw muscles in particular, which occur particularly in the case of chronic bruxism sufferers, are found to be able to suffice with a relatively low muscle activity to nevertheless bring about an adverse clenching. This means that the device must already generate a correcting stimulus at a relatively low threshold value. Normal or weak jaw muscles on the other hand require a relatively high jaw muscle activity before there is any danger of damage to the jaws. With early or even preventive use of the device the threshold value must therefore be relatively high. To enable optimal adjustment of the device to the individual user, the bio-feedback system according to the invention is provided with means for adjusting and storing the threshold value.

The means for adjusting the threshold value can be embodied in diverse ways. In the first place it is possible to arrange a variable resistor or variable capacitor in an electronic circuit in which the control unit is incorporated so as to thereby enable manual setting and storing of a reference value from which the threshold level ensues. In the exemplary embodiment described here, use is made of a fully integrated software approach. For this purpose the control unit is set in a learning mode in accordance with a predetermined instruction, whereafter the output signal of the user is detected. A running average of the rest level is thus determined first of all and stored as offset value V_{off} in a non-volatile electronically rewritable memory of the control unit. The running average of the output signal of the signal sensor is subsequently determined during powerful clenching together of the jaws as well as during grinding of the jaws. These two values are also stored in the memory as first and second threshold value V₁ and V₂ respectively. A threshold value is thus individually determined per user which takes account of the individual state, and particularly the specific jaw muscle activity. The device herein accepts threshold values between 5 millivolts and 50 millivolts, but can if desired be modified for a wider adjustment range.

During operation the control unit continuously receives and then analyses an output signal from signal sensor 2 (IN), see FIG. 3. The control unit herein distinguishes signals in a first frequency domain up to about 20 Hz and signals received in a second frequency domain from about 90 Hz to 600 Hz. The measured signal is carried through a differential amplifier and then split in a separating filter 31 into a low-frequency (LF) and a high-frequency (HF) component. Both components are filtered, each with its own band filter 32,33 in accordance with the above stated frequency ranges, in order to eliminate noise and other interference signals as far as possible. Both signal are finally made suitable for digitizing. This takes place by amplifying the signal and adding a fixed offset thereto. Both resulting signals are then digitized with a 12-bit analog-digital converter (ADC) 34,35 with a sampling frequency of 1024 Hz for both the low-frequency (LF) and the high-frequency (HF) signal.

Both the LF and the HF signal have an operational offset depending on factors such as the temperature and the supply voltage. In order to compensate the signal herefor, this offset is determined in the rest state and stored as such in the memory. In the order of 2̂16=65536 samples are taken for this purpose and the average value thereof stored as offset. This calibration is performed each time the device is taken into use and is adjusted dynamically.

During operation the average net signal strength V_{LF}, V_{HF} respectively of both signals is determined relative to the offset value (V_{offset}) in accordance with the following formula:

$${V}_{LF} = {\sum }_{i = 0}^{n} \frac{‖ {V}_{i} - {V}_{offset} ‖}{n}$$

$${V}_{HF} = {\sum }_{i = 0}^{n} \frac{‖ {V}_{i} - {V}_{offset} ‖}{n}$$

wherein n is the number of samples taken and Vᵢ is the signal strength of a taken sample. The higher the number of samples, the more stable is the measured signal strength and the slower this algorithm responds to incidental fluctuations in the measured signal strength. The device described here makes use of at least 2̂6=64 samples for the HF signal en 2̂10=1024 samples for the LF signal. These values are found in practice to produce reliable measurements. The choice for a power of two in respect of the sample number enhances the efficiency of a binary processing of the algorithm by the control unit.

The measured signal strength is a measure of the momentary muscle activity in the jaw system of the user. The invention is here based on the insight that strongly developed muscles display less activity than weakly developed jaw muscles to develop a comparable muscle power. Users who already suffer to a greater or lesser extent from bruxism generally have relatively strongly developed jaw muscles as a result. Users who employ the device preventively will on the other hand generally have relatively less developed jaw muscles. The device according to the invention takes this into account by establishing an individual threshold value and thus adjusting the device to the user. In this example these are the first and second threshold value V₁, V₂ which respectively represent the actual grinding of the teeth and clenching of the jaws. Because these values have been operationally determined and stored, these values will usually be lower for a user with bruxism than for a user who employs the device preventively.

As soon as a muscle activity above the threshold value is detected for a predetermined time period of 2 seconds, which is otherwise adjustable between 1 and 5 seconds, the control unit will interpret this as bruxism and generate a correcting stimulus via electrodes 2. This time interval can otherwise be adjusted separately for both the LF signal and the HF signal, so that in this respect too the device can be adjusted optimally. Setting of the time interval takes place via factory software settings or by a registered service engineer.

Because the measured measurement signal is not always perfectly flat and continuous, the control unit takes account of fluctuations in the signal strength. The control unit comprises for this purpose adding means in the form of a counter 36,37 which adds as long as the signal is above the threshold value and subtracts during the time the signal strength drops below it. The counter frequency is also adjustable and set in this case at 1000 Hz. When the signal strength of either the low-frequency signal or the high-frequency signal is detected continuously for longer than the relevant time interval, of in this case 2 seconds in both cases, above the respective threshold value, the control unit will interpret this as a symptom of bruxism and generate a correcting stimulus. The same applies however when the signal strength persists for instance for only one and a half seconds above the threshold value, then falls below it for instance for half a second and subsequently rises above the threshold value again for a minimum of a second. In this case too there is in sum a signal strength above the threshold value for more than the set interval of 2 seconds.

When the time interval of either the LF signal or the HF signal above the threshold value is exceeded, the relevant adding means will generate a logic high signal to a logic gate 38. This gate 38 is coupled to a pulse generator 39 which is thus actuated as soon as one of the two inputs of the OR gate 38 receives a high value. Pulse generator 39 then generates a suitable correcting stimulus which is transmitted via electrode 2 to the jaw of the user.

The invention thus provides a device for preventing bruxism which is closely linked to the individual physical condition of the user so as to thus be able to intervene optimally when bruxism occurs.

Although the invention has been further elucidated above with reference to only a single exemplary embodiment, it will be apparent that the invention is by no means limited thereto. On the contrary, many variations and embodiments are still possible within the scope of the invention for a person with ordinary skill in the art.

In the shown example use is thus made of detection of a low-frequency signal and a high-frequency signal to deal with different forms of bruxism, each with its own threshold level and time duration, although an embodiment also lies within the scope of the invention wherein measurement takes place in only one of the two ranges or a shared threshold level and/or time duration is applied. In the given embodiment use is made of mutually differing, non-overlapping frequency domains to detect different forms of bruxism. It is however possible instead in some cases for both frequency domains to be partially overlapping or to even wholly coincide, in accordance with the circumstances of the user.

The device can in some cases be provided with means to suppress disruptive ambient influences. Measures can thus be taken to eliminate, or at least adequately suppress, 50 Hz noise signals, which signals usually come from electrical equipment connected to mains electricity. In the case of mains electricity with an alternating voltage at a different frequency, this also applies mutatis mutandis for this different frequency.

The threshold value and identification value applied in the exemplary embodiment can be stored in the carrier as well as outside the carrier, in which case the electronic components required for this purpose are integrated therein. The carrier can thus be adjusted to the user in wholly individual manner both mechanically, in shape and fit, and electronically, in terms of the threshold value included therein, which then even provides the option of producing a wholly uniform control unit, since all personal parameters required for actuation are incorporated in the carrier itself and can be read therefrom.

1. Device for preventing bruxism, comprising a carrier intended for receiving in a mouth of a user, which carrier comprises at least a part of an electronic bio-feedback system, which bio-feedback system comprises at least one signal sensor which is sensitive to electrical muscle signals and which is coupled to a control unit designed and adapted to generate a correcting stimulus via an electrode when a muscle signal is detected above a predetermined threshold level, characterized in that the bio-feedback system is provided with means for adjusting and storing the threshold level.

2. Device as claimed in claim 1, characterized in that the threshold value is adjustable to a level of between about 5 millivolts and 50 millivolts.

3. Device as claimed in claim 1, characterized in that the bio-feedback system is provided with means for placing the device in a learning mode in which an output signal from the signal sensor is stored as threshold signal.

4. Device as claimed in claim 1, characterized in that the at least one signal sensor is able and adapted to detect a first muscle signal in a first frequency domain and a second muscle signal in a second frequency domain, and that the bio-feedback system comprises a threshold value per frequency domain.

5. Device as claimed in claim 4, characterized in that the first and second frequency domain lie respectively in a frequency range of about 1 to 20 Hz and about 90 to 600 Hz.

6. Device as claimed in claim 1, characterized in that the control unit comprises a processing unit which is able and adapted to determine a running average of a muscle signal measured during a rest state and to store this average as offset value.

7. Device as claimed in claim 6, characterized in that the processing unit is able and adapted to decrease a measured muscle signal by the offset value in an operating state and present this as usable signal to the control unit.

8. Device as claimed in claim 1, characterized in that the control unit comprises clock means for generating a control signal only after a determined time period has elapsed during which the input signal lies above said threshold value.

9. Device as claimed in claim 8, characterized in that said time period is adjustable between 1 and 5 seconds.

10. Device as claimed in claim 8, characterized in that the control unit comprises adding means which are able and adapted to find the sum of time periods during which said threshold value has been exceeded while subtracting intervening periods during which said threshold value has not been reached.

11. Device as claimed in claim 1, characterized in that the carrier is provided with an electronic element which is able to comprise an electronically detectable identification value associated with the carrier, and that the control device comprises read means which are able to record the identification value of a carrier coupled thereto and compare it to a value stored in the control device.

12. Device as claimed in claim 11, characterized in that the electronic element is from a group comprising electrical resistors, capacitors and memory circuits which can at least be read out electronically.

13. Device as claimed in claim 11, characterized in that the carrier is connected by means of a cable to the control unit and that the electronic element is integrated in the cable, in particular in a connector thereof.

14. Device as claimed in claim 2, characterized in that the bio-feedback system is provided with means for placing the device in a learning mode in which an output signal from the signal sensor is stored as threshold signal.

15. Device as claimed in claim 2, characterized in that the at least one signal sensor is able and adapted to detect a first muscle signal in a first frequency domain and a second muscle signal in a second frequency domain, and that the bio-feedback system comprises a threshold value per frequency domain.

16. Device as claimed in claim 3, characterized in that the at least one signal sensor is able and adapted to detect a first muscle signal in a first frequency domain and a second muscle signal in a second frequency domain, and that the bio-feedback system comprises a threshold value per frequency domain.

17. Device as claimed in claim 9, characterized in that the control unit comprises adding means which are able and adapted to find the sum of time periods during which said threshold value has been exceeded while subtracting intervening periods during which said threshold value has not been reached.

18. Device as claimed in claim 12, characterized in that the carrier is connected by means of a cable to the control unit and that the electronic element is integrated in the cable, in particular in a connector thereof.
